(19) United States
(12) Patent Application Publication
(10) Pub. No.: US 2005/0266089 A1
(43) Pub. Date: Dec. 1, 2005

(54) Contact lens and eye drop rewetter compositions and methods

(76) Inventors: James N. Cook, Mission Viejo, CA (US); Stanley W. Huth, Newport Beach, CA (US)

Correspondence Address: ADVANCED MEDICAL OPTICS, INC., 1700 E. ST. ANDREW PLACE, SANTA ANA, CA, 92705, US

(21) Appl. No.: 11/192,718

(22) Filed: Jul. 29, 2005

Related U.S. Application Data
(63) Continuation of application No. 10/752,759, filed on Jan. 7, 2004.
(60) Provisional application No. 60/438,857, filed on Jan. 8, 2003.
(60) Provisional application No. 60/438,843, filed on Jan. 8, 2003.

(51) Int. Cl. A61K 33/14; A61K 31/728

(52) U.S. Cl. 424/488; 424/661; 514/54

(57) ABSTRACT

Stable ophthalmic formulations comprising hyaluronic acid (sodium hyaluronate) as the primary active demulcent ingredient, stabilized oxy-chloro complex (available commercially as OcuPure™ from Advanced Medical Optics, Purite® from Allergan, and Purogene from Biocide) for preservative efficacy, balanced salts mimicking the tear film, and sodium borate as a buffer are disclosed. In one embodiment, preferred stable formulations may be used in the human eye with or without contact lenses. In another embodiment preferred formulations may also be used as a storage and conditioning solution for contact lenses following disinfection.

### RELATED APPLICATION DATA

This application claims priority under 35 U.S.C. 119(e) to Provisional Applications Nos. 60/438,857 and 60/438,843, both filed Jan. 8, 2003. The disclosures of these provisional applications are incorporated in their entirety herein by reference.

### BACKGROUND OF THE INVENTION

1. Field of the Invention

This invention relates generally to a rewetter formulation suitable for use in the human eye. The rewetter formulation may be used in human eyes with and without contact lenses. Additionally, this formulation can be used as a storage or conditioning solution for contact lenses following disinfection. More particularly, preferred formulations provide superior initial and long lasting comfort to contact lens wearers experiencing dryness and irritation.

2. Description of the Related Art

Contact lenses provide a valuable option to the vision impaired. Although there have been vast improvements in the materials used for contact lenses, irritation due to use of these lenses still remains. Often wearers experience dry itchy eyes due to moisture loss in the contact lens. This can be compounded by environmental pollutants and associated allergies. Irritation can also be caused by particles that adhere to the lens. In order to continue use of the lenses, users often resort to rewetting solutions. These solutions are used to rehydrate the contact lens thereby increasing comfort to the wearer. They can also be used to remove particulate matter from the surface of the lens and to store the lens if necessary. These solutions can also be used by people who suffer from dry eye symptoms and do not wear contact lenses.

As these solutions are used in the eye, they must be sterile and free of irritating contaminants. Many known preservatives are unfortunately unsuitable for use in the eye. It is necessary to find a preservative that is effective yet non-irritating. Further, it is useful if the rewetting solution has antimicrobial activity. The minimum antimicrobial activity necessary should ensure that there is substantially no increase in microorganisms in the rewetting solution or in the eye. This helps to ensure that the user does not suffer from unnecessary eye infections or irritation.

In addition to rewetting, there is also a need for storage and conditioning solutions with similar properties.

There continues to be a need for rewetting, storage, and conditioning solutions that provide increased comfort to the eye.

### SUMMARY OF THE INVENTION

In accordance with one embodiment, preferred stable rewetter formulations comprising hyaluronic acid (sodium hyaluronate) as the primary active demulcent ingredient, stabilized oxy-chloro complex (available commercially as as OcuPure™ from Advanced Medical Optics, Purite® from Allergan, and Purogene from Biocide) for preservative efficacy, and sodium borate as a buffer are disclosed. In other embodiments, preferred stable formulations further comprise balanced salts mimicking the tear film and/or additional demulcents. In one embodiment, preferred stable formulations may be used in the human eye with or without contact lenses. For example, preferred stable formulations may be used to treat the symptoms of dry eye. In another embodiment preferred stable formulations may also be used as a storage and conditioning solution for contact lenses following disinfection.

In one embodiment wherein hyaluronic acid is the primary active demulcent, the hyaluronic acid preferably has a molecular weight of about 200,000 to about 4,000,000 daltons. Preferably, the range is from about 750,000 to about 2,000,000 daltons. More preferably, the range is from about 800,000 to about 1,750,000 daltons. An even more preferred range is from about 900,000 to about 1,500,000 daltons. In a preferred embodiment the concentration of hyaluronic acid is from about 0.005% to about 0.5% weight/volume (w/v). Preferably the hyaluronic acid concentration ranges from about 0.01 to about 0.3% w/v. In a more preferred embodiment the hyaluronic acid concentration ranges from about 0.02 to about 0.2% w/v. In another preferred embodiment the concentration of hyaluronic acid is from about 0.05% to about 2% w/v, more preferably from about 0.1 to about 0.5% w/v, but also including about 0.2, 0.4, 0.6, 0.8, 1.2, 1.4, 1.6, and 1.8% w/v. Preferably the stabilized oxy-chloro complex concentration ranges from about 0.0015 to about 0.05% w/v. More preferably the stabilized oxy-chloro complex concentration ranges from about 0.002 to about 0.04% w/v. More preferably the stabilized oxy-chloro complex concentration ranges from about 0.0025 to about 0.03% w/v. Another preferred stabilized oxy-chloro complex concentration ranges from about 0.003 to about 0.02% w/v. In a further preferred embodiment, the stabilized oxy-chloro complex concentration ranges from about 0.0035 to about 0.01% w/v. More preferably the stabilized oxy-chloro complex concentration ranges from about 0.004 to about 0.009% w/v. One preferred embodiment has a pH range of about 6.0 to about 9.0, preferably from about 6.8 to about 8.0, more preferably from about 7.0 to about 7.4, with the most preferred pH of approximately 7.2. To maintain this pH, a buffer solution of boric acid and sufficient borate salt, with suitable counterions, is added.

In one embodiment, a preferred stable formulation further comprises balanced salts. The balanced salts of certain embodiments preferably include NaCl, KCl, CaCl₂, and MgCl₂ in a ratio that provides an osmolality range of about 140 to about 400, preferably about 240 to about 330 mOsm/kg, preferably about 260 to about 300 mOsm/kg, with the most preferred osmolality of approximately 270 mOsm/kg. In one embodiment, NaCl ranges from about 0.1 to about 1% w/v, preferably from about 0.2 to about 0.8% w/v, more preferably about 0.39% w/v, KCl ranges from about 0.02 to about 0.5% w/v, preferably about 0.05 to about 0.3% w/v, more preferably about 0.14% w/v, CaCl₂ ranges from about 0.0005 to about 0.1% w/v, preferably about 0.005 to about 0.08% w/v, more preferably about 0.06% w/v, and MgCl₂ ranges from about 0.0005 to about 0.1% w/v, preferably about 0.005 to about 0.08% w/v, more preferably about 0.06% w/v.

In one embodiment, a preferred stable formulation further comprises additional demulcents. Suitable additional demulcents include, but are not limited to, cellulose derivatives ranging from about 0.2 to about 2.5 percent such as carboxymethylcellulose sodium, hydroxyethyl cellulose, hydroxypropyl methylcellulose, and methylcellulose; gelatin at about 0.01%; polyols in about 0.05 to about 1%, also including about 0.2 to about 1%, such as glycerin, polyethylene glycol 300, polyethylene glycol 400, polysorbate 80, and propylene glycol; polyvinyl alcohol from about 0.1 to about 4 percent; povidone from about 0.1 to about 2%; and dextran 70 from about 0.1% when used with another polymeric demulcent described herein. Of these additional demulcents, in certain embodiments, polyols are particularly preferred. In other embodiments, cellulose derivatives are also preferred. Preferred cellulose derivatives preferably have a molecular weight equal to or less than about 80,000, more preferably about 10,000 to about 40,000. In certain circumstances, demulcents with large molecular weights could negatively affect preferred formulations.

In another embodiment, preferred stable rewetter formulations are instilled into the human eye to treat dry eye symptoms. In preferred embodiments stable formulations may be instilled into eyes with and without contact lenses. In one embodiment wherein hyaluronic acid is the primary active demulcent, the hyaluronic acid preferably has a molecular weight of about 200,000 to about 4,000,000 daltons. Preferably, the range is from about 750,000 to about 2,000,000 daltons. More preferably, the range is from about 800,000 to about 1,750,000 daltons. An even more preferred range is from about 900,000 to about 1,500,000 daltons. In a preferred embodiment the concentration of hyaluronic acid is from about 0.005% to about 0.5% weight/volume (w/v). Preferably the hyaluronic acid concentration ranges from about 0.01 to about 0.3% w/v. In a more preferred embodiment the hyaluronic acid concentration ranges from about 0.02 to about 0.2% w/v. In another preferred embodiment the concentration of hyaluronic acid is from about 0.05% to about 2% w/v, more preferably from about 0.1 to about 0.5% w/v, but also including about 0.2, 0.4, 0.6, 0.8, 1.2, 1.4, 1.6, and 1.8% w/v. Preferably the stabilized oxy-chloro complex concentration ranges from about 0.0015 to about 0.05% w/v. More preferably the stabilized oxy-chloro complex concentration ranges from about 0.002 to about 0.04% w/v. More preferably the stabilized oxy-chloro complex concentration ranges from about 0.0025 to about 0.03% w/v. Another preferred stabilized oxy-chloro complex concentration ranges from about 0.003 to about 0.02% w/v. In a further preferred embodiment, the stabilized oxy-chloro complex concentration ranges from about 0.0035 to about 0.01% w/v. More preferably the stabilized oxy-chloro complex concentration ranges from about 0.004 to about 0.009% w/v. One preferred embodiment has a pH range of about 6.0 to about 9.0, preferably from about 6.8 to about 8.0, more preferably from about 7.0 to about 7.4, with the most preferred pH of approximately 7.2. To maintain this pH, a buffer solution of boric acid and sufficient borate salt, with suitable counterions, is added.

In one embodiment, a preferred stable formulation further comprises balanced salts. The balanced salts of certain embodiments preferably include NaCl, KCl, CaCl₂, and MgCl₂ in a ratio that provides an osmolality range of about 240 to about 330 mOsm/kg, preferably about 260 to about 300 mOsm/kg, with the most preferred osmolality of approximately 270 mOsm/kg.

In one embodiment, a preferred stable formulation further comprises additional demulcents. Suitable additional demulcents include, but are not limited to, cellulose derivatives ranging from about 0.2 to about 2.5 percent such as carboxymethylcellulose sodium, hydroxyethyl cellulose, hydroxypropyl methylcellulose, and methylcellulose; gelatin at about 0.01%; polyols in about 0.05 to about 1%, also including about 0.2 to about 1%, such as glycerin, polyethylene glycol 300, polyethylene glycol 400, polysorbate 80, and propylene glycol; polyvinyl alcohol from about 0.1 to about 4 percent; povidone from about 0.1 to about 2%; and dextran 70 from about 0.1% when used with another polymeric demulcent described herein. Of these additional demulcents, in certain embodiments, polyols are particularly preferred. In other embodiments, cellulose derivatives are also preferred. Preferred cellulose derivatives preferably have a molecular weight equal to or less than about 80,000, more preferably about 10,000 to about 40,000. In certain circumstances, demulcents with large molecular weights could negatively affect preferred formulations.

All of these embodiments are intended to be within the scope of the invention herein disclosed. These and other embodiments of the present inventions will become readily apparent to those skilled in the art from the following detailed description of preferred embodiments, the invention not being limited to any particular preferred embodiment(s) disclosed.

### DETAILED DESCRIPTION OF THE PREFERRED EMBODIMENT

Disclosed herein is a new stable ophthalmic formulation useful as a rewetter. Broadly one preferred embodiment is a stable combination that includes hyaluronic acid (sodium hyaluronate) as the primary active demulcent ingredient, stabilized oxy-chloro complex for preservative efficacy, and sodium borate/boric acid as a buffer. Preferred embodiments may further comprise balanced salts mimicking the tear film and/or additional demulcents. Hyaluronic acid was selected as the demulcent to provide superior initial and long lasting comfort to contact lens wearers experiencing dryness and irritation. The viscoelastic, lubrication and water-retaining properties of hyaluronic acid are well known and are superior to cellulose-derived demulcents such as hydroxypropylmethylcellulose (HPMC) and carboxymethylcellulose (CMC). A unique property of hyaluronic acid is that it resembles tear mucus by maintaining viscosity between blinks, but undergoes shear-thinning during blinks. This property enhances residence time, maintaining water on and around the lens, providing superior cushioning and relief from dryness and irritation associated with contact lens wear.

As used herein, the term “demulcent” is a broad term used in its ordinary sense and includes embodiments wherein “demulcent” also refers to, without limitation, an agent, usually a water soluble polymer, which is applied topically to the eye to protect and lubricate mucous membrane surfaces and relieve dryness and irritation. As used herein, the term “stable formulation” is a broad term used in its ordinary sense and includes embodiments wherein “stable formulation” also refers to embodiments wherein the viscosity of preferred formulations experiences a viscosity breakdown of less than or equal to about 70% over 12 months at 25° C., more preferably less than or equal to about 50% over 12′ months at 25° C. Although embodiments disclosed herein may be in terms of contact lens use, one of skill in the art will recognize that preferred embodiments may also be used in humans who are not wearing contact lenses.

As used herein, the term “stabilized oxy-chloro complex” is a broad term used in its ordinary sense. The term includes, without limitation, a stable solution comprising a chlorine dioxide precursor or to a chlorine dioxide precursor with chlorine dioxide in equilibrium. Chlorine dioxide precursors include, but are not limited to, chlorite components such as metal chlorites, for example alkali metal and alkaline earth metal chlorites. One particularly preferred metal chlorite is sodium chlorite. Stabilized oxy-chloro complex as stabilized chlorine dioxide is available commercially as OCUPURE™ from Advanced Medical Optics, PURITE® from Allergan, and PUROGENE from Biocide.

As used herein, concentrations of stabilized oxy-chloro complex are measured in terms of potential chlorine dioxide. As used herein, the term “potential chlorine dioxide” is a broad term used in its ordinary sense. As such, one sense of the term refers to the amount of chlorine dioxide potentially provided if all chlorine dioxide precursor, such as sodium chlorite, were converted to chlorine dioxide. One way to convert sodium chlorite to chlorine dioxide is to dissolve the sodium chlorite and acidify the resulting solution. Although, other manners of conversion are well known to those skilled in the art, including exposure to transition metals.

One of skill in the art would expect that the addition of stabilized oxy-chloro complex to hyaluronic acid would result in a greater decrease in viscosity than formulas containing hyaluronic acid without purite. Those of skill in the art would expect that the oxy-chloro complex radical would react with the hyaluronate subunit sidechain thereby cleaving the bond between subunits. Thus, those of skill in the art would have expected that this polymer chain cleavage would cause a more dramatic decrease in viscosity when compared to formulas with hyaluronic acid alone. However, unexpectedly, the preferred formulations comprising hyaluronic acid and stabilized oxy-chloro complex provide viscosity stability. As discussed below in Example 2, a direct comparison of two formulations, one with stabilized oxy-chloro complex and one without stabilized oxy-chloro complex demonstrated that the viscosity of the formula containing stabilized oxy-chloro complex was surprisingly similar to the formula without purite.

The purite/borate disinfection and buffer system is ideal for preferred formulations. This system has been proven to yield good preservative efficacy against bacteria, yeast and fungi, yet is mild to mammalian cells. Additionally, the stabilized oxy-chloro complex preservative is negatively charged ensuring compatibility with the negatively charged hyaluronic acid demulcent.

An advantage of the purite/borate system over perborate or hydrogen peroxide systems is that both perborate and hydrogen peroxide can irritate the eye. When perborate is dissolved in water, hydrogen peroxide is formed which can cause eye irritation. Hydrogen peroxide at levels of 0.01% and higher has been shown to cause discomfort in the eye. See Paugh, J., Brennan, N., and Efron, N., “Ocular Response to Hydrogen Peroxide,” Am J Optom Physiol Opt. 1988 February;65(2):91-8. Thus, preferred embodiments of the present composition have less than 0.01% hydrogen peroxide, more preferably less than about 0.0075% hydrogen peroxide, still more preferably less than about 0.005% hydrogen peroxide, and most preferably hydrogen peroxide is substantially absent. These preferred embodiments also have less than the amount of any component, such as perborate, that will release hydrogen peroxide to produce 0.01% hydrogen peroxide, more preferably less than about 0.0075% hydrogen peroxide, and still more preferably less than about 0.005% hydrogen peroxide.

Most preferably, hydrogen peroxide or components that release hydrogen peroxide are substantially absent. Many commercially available stabilized oxy-chloro compositions contain insubstantial amounts of peroxide as impurities. For example, the product sold under the trade name PUROGENE by Biocide may contain an insubstantial amount of hydrogen peroxide, up to 0.002% peroxide, in a 2% solution. Accordingly, a preferred embodiment of the present composition utilizing the PUROGENE product may contain up to 0.00003% peroxide even without the addition of hydrogen peroxide or compounds that release hydrogen peroxide.

Advantageously when the purite/borate system reacts with the water in the eye without the presence of hydrogen peroxide, only salt and oxygen are formed. The oxygen dissipates without causing irritation to the eye, and can advantageously alleviate hypoxic conditions in the eye.

One preferred formulation includes, but is not limited to, NaCl, KCl, CaCl₂, and MgCl₂ balanced salts which mimic the mineral composition of tears. This provides additional enhanced comfort and relieves irritation through replacement of any essential salts that may be reduced during lens wear. This is preferred to NaCl alone as NaCl alone can actually cause eye stress. Therefore the disclosed combination is preferable.

Unexpectedly the combination of hyaluronic acid, stabilized oxy-chloro complex and the borate buffer system results in increased comfort, as well as other advantages. For example, as discussed below in the Examples section, when compared with a commercially available eye drop, Refresh, preferred formulations provided an increased length of comfort effect after using drops, greater comfort at the end of the day, improved tear break-up time, and longer lens wearing time during the day due to the enhanced comfort provided when compared to Refresh.

It is believed that preferred formulations of certain embodiments are less cytotoxic than other marketed rewetter compositions resulting in greater comfort. In addition, preferred formulations provide superior wettability. Enhanced wettability translates clinically to expected enhancement of comfort and longer duration of wear. Therefore, preferred formulations not only provide superior comfort to contact lens wearers suffering dryness and irritation associated with lens wear, but also provide longer duration of wear.

It is believed that preferred formulations of certain embodiments will neutralize positively charged antimicrobials and preservatives commonly used in contact lens disinfecting solutions thereby enhancing comfort. This is especially helpful for lens wearers who are allergic or sensitive to these positively charged antimicrobials and preservatives. In one embodiment the antimicrobial or preservative is neutralized by contacting the preferred formulation with the contact lens while the lens is in the eye. Alternatively, preferred formulations may be contacted with the lens outside the eye by placing several drops of solution on the lens or by using the solution as a storage or conditioning solution after disinfection.

In one embodiment a preferred stable formulation comprises hyaluronic acid (sodium hyaluronate) as the primary active demulcent ingredient, stabilized oxy-chloro complex for preservative efficacy, and sodium borate/boric acid as a buffer. Preferred embodiments may further comprise balanced salts mimicking the tear film and/or an additional demulcent. In one embodiment, the hyaluronic acid preferably has a molecular weight of about 200,000 to about 4,000,000 daltons. Preferably, the range is from about 750,000 to about 2,000,000 daltons. More preferably, the range is from about 800,000 to about 1,750,006 daltons. An even more preferred range is from about 900,000 to about −1,500,000 daltons. In a preferred embodiment the concentration of hyaluronic acid is from about 0.005% to about 0.5% weight/volume (w/v). Preferably the hyaluronic acid concentration ranges from about 0.01 to about 0.3% w/v. In a more preferred embodiment the hyaluronic acid concentration ranges from about 0.02 to about 0.2% w/v. In another preferred embodiment the concentration of hyaluronic acid is from about 0.05% to about 2% w/v, more preferably from about 0.1 to about 0.5% w/v, but also including about 0.2, 0.4, 0.6, 0.8, 1.2, 1.4, 1.6, and 1.8% w/v. Preferably the stabilized oxy-chloro complex concentration ranges from about 0.0015 to about 0.05% w/v. More preferably the stabilized oxy-chloro complex concentration ranges from about 0.002 to about 0.04% w/v. More preferably the stabilized oxy-chloro complex concentration ranges from about 0.0025 to about 0.03% w/v. Another preferred stabilized oxy-chloro complex concentration ranges from about 0.003 to about 0.02% w/v. In a further preferred embodiment, the stabilized oxy-chloro complex concentration ranges from about 0.0035 to about 0.01% w/v. More preferably the stabilized oxy-chloro complex concentration ranges from about 0.004 to about 0.009% w/v. One preferred embodiment has a pH range of about 6.0 to about 9.0, preferably from about 6.8 to about 8.0, more preferably from about 7.0 to about 7.4, with the most preferred pH of approximately 7.2. To maintain this pH, a buffer solution of boric acid and sufficient borate salt, with suitable counterions, is added.

In one embodiment, a preferred stable formulation further comprises balance salts. The balanced salts of certain embodiments preferably include NaCl, KCl, CaCl₂, and MgCl₂ in a ratio that provides an osmolality range of about 140 to about 400 mOsm/kg, preferably about 240 to about 330 mOsm/kg, preferably about 260 to about 300 mOsm/kg, with the most preferred osmolality of approximately 270 mOsm/kg. In one embodiment, NaCl ranges from about 0.1 to about 1% w/v, preferably from about 0.2 to about 0.8% w/v, more preferably about 0.39% w/v, KCl ranges from about 0.02 to about 0.5% w/v, preferably about 0.05 to about 0.3% w/v, more preferably about 0.14% w/v, CaCl₂ ranges from about 0.0005 to about 0.1% w/v, preferably about 0.005 to about 0.08% w/v, more preferably about 0.06% w/v, and MgCl₂ ranges from about 0.0005 to about 0.1% w/v, preferably about 0.005 to about 0.08% w/v, more preferably about 0.06% W/V.

In one embodiment, a preferred stable formulation further comprises additional demulcents. Additional demulcents include, but are not limited to, the approved ophthalmic demulcents described in the United States Ophthalmic Demulcents Monograph. See 21 CFR 349.12 (2003). Suitable additional demulcents include, but are not limited to, cellulose derivatives ranging from about 0.2 to about 2.5 percent such as carboxymethylcellulose sodium, hydroxyethyl cellulose, hydroxypropyl methylcellulose, and methylcellulose; gelatin at about 0.01%; polyols in about 0.05 to about 1%, also including about 0.2 to about 1%, such as glycerin, polyethylene glycol 300, polyethylene glycol 400, polysorbate 80, and propylene glycol; polyvinyl alcohol from about 0.1 to about 4 percent; povidone from about 0.1 to about 2%; and dextran 70 from about 0.1% when used with another polymeric demulcent described herein. Of these additional demulcents, in certain embodiments, polyols are particularly preferred. In other embodiments, cellulose derivatives are also preferred. Preferred cellulose derivatives preferably have a molecular weight equal to or less than about 80,000, more preferably about 10,000 to about 40,000. In certain circumstances, demulcents with large molecular weights could negatively affect preferred formulations.

In another embodiment, preferred stable formulations are instilled into the human eye to treat dry eye symptoms. In another embodiment, preferred stable formulations are instilled into a mammal's eye to treat dry eye symptoms. In preferred embodiments formulations may be instilled into eyes with and without contact lenses. In one embodiment a preferred stable formulation comprises hyaluronic acid (sodium hyaluronate) as the primary active demulcent ingredient, stabilized oxy-chloro complex for preservative efficacy, and sodium borate/boric acid as a buffer. Preferred embodiments may further comprise balanced salts mimicking the tear film and/or another demulcent. In one embodiment the hyaluronic acid preferably has a molecular weight of about 200,000 to about 4,000,000 daltons. Preferably, the range is from about 750,000 to about 2,000,000 daltons. More preferably, the range is from about 800,000 to about 1,750,000 daltons. An even more preferred range is from about 900,000 to about 1,500,000 daltons. In a preferred embodiment the concentration of hyaluronic acid is from about 0.005% to about 0.5% weight/volume (w/v). Preferably the hyaluronic acid concentration ranges from about 0.01 to about 0.3% w/v. In a more preferred embodiment the hyaluronic acid concentration ranges from about 0.02 to about 0.2% w/v. In another preferred embodiment the concentration of hyaluronic acid is from about 0.05% to about 2% w/v, more preferably from about 0.1 to about 0.5% w/v, but also including about 0.2, 0.4, 0.6, 0.8, 1.2, 1.4, 1.6, and 1.8% w/v. Preferably the stabilized oxy-chloro complex concentration ranges from about 0.0015 to about 0.05% w/v. More preferably the stabilized oxy-chloro complex concentration ranges from about 0.002 to about 0.04% w/v. More preferably the stabilized oxy-chloro complex concentration ranges from about 0.0025 to about 0.03% w/v. Another preferred stabilized oxy-chloro complex concentration ranges from about 0.003 to about 0.02% w/v. In a further preferred embodiment, the stabilized oxy-chloro complex concentration ranges from about 0.0035 to about 0.01% w/v. More preferably the stabilized oxy-chloro complex concentration ranges from about 0.004 to about 0.009% w/v. One preferred embodiment has a pH range of about 6.0 to about 9.0, preferably from about 6.8 to about 8.0, more preferably from about 7.0 to about 7.4, with the most preferred pH of approximately 7.2. To maintain this pH, a buffer solution of boric acid and sufficient borate salt, with suitable counterions, is added.

In one embodiment, a preferred stable formulation further comprises balance salts. The balanced salts of certain embodiments preferably include NaCl, KCl, CaCl₂, and MgCl₂ in a ratio that provides an osmolality range of about 140 to about 400 mOsm/kg, preferably about 240 to about 330 mOsm/kg, preferably about 260 to about 300 mOsm/kg, with the most preferred osmolality of approximately 270 mOsm/kg. In one embodiment, NaCl ranges from about 0.1 to about 1% w/v, preferably from about 0.2 to about 0.8% w/v, more preferably about 0.39% w/v, KCl ranges from about 0.02 to about 0.5% w/v, preferably about 0.05 to about 0.3% w/v, more preferably about 0.14% w/v, CaCl₂ ranges from about 0.0005 to about 0.1% w/v, preferably about 0.005 to about 0.08% W/V, more preferably about 0.06% w/v, and MgCl₂ ranges from about 0.0005 to about 0.1% w/v, preferably about 0.005 to about 0.08% w/v, more preferably about 0.06% w/v.

In one embodiment, a preferred stable formulation further comprises additional demulcents. Additional demulcents include, but are not limited to, the approved ophthalmic demulcents described in the United States Ophthalmic Demulcents Monograph. See 21 CFR 349.12 (2003). Suitable additional demulcents include, but are not limited to, cellulose derivatives ranging from about 0.2 to about 2.5 percent such as carboxymethylcellulose sodium, hydroxyethyl cellulose, hydroxypropyl methylcellulose, and methylcellulose; gelatin at about 0.01%; polyols in about 0.05 to about 1%, also including about 0.2 to about 1%, such as glycerin, polyethylene glycol 300, polyethylene glycol 400, polysorbate 80, and propylene glycol; polyvinyl alcohol from about 0.1 to about 4 percent; povidone from about 0.1 to about 2%; and dextran 70 from about 0.1% when used with another polymeric demulcent described herein. Of these additional demulcents, in certain embodiments, polyols are particularly preferred. In other embodiments, cellulose derivatives are also preferred. Preferred cellulose derivatives preferably have a molecular weight equal to or less than about 80,000, more preferably about 10,000 to about 40,000. In certain circumstances, demulcents with large molecular weights could negatively affect preferred formulations.

Preferred formulations are prepared using standard compounding, filtration, fill and packaging equipment. In one embodiment preferred formulations are prepared in a scaled up version capable of mass production. In another embodiment preferred formulations are prepared in small laboratory scale batches. In one embodiment the packaging used consists of single use containers. In some single use embodiments, an alternative formulation may include non-preserved formulations. The non-preserved embodiments may also replace the borate/boric acid buffer system with a milder buffer system such as about 0.3% sodium lactate. In another embodiment, the formulation is packaged in eye dropper bottles of varying sizes. In another embodiment the solution is packaged in bottles of suitable size for use of the formula as a contact lens storage or conditioning solution. Preferred packaging includes, but is not limited to, materials that will shield the invention from light. One embodiment of the packaging consists of teal bottles. Other embodiments include bottles of various colors, for example blue, opaque white, black, or brown bottles can be used.

The following detailed examples are illustrations of preferred embodiments. It should be clear that these are not intended to limit the scope of the present invention.

### EXAMPLE 1

The following is an example of a preferred single demulcent embodiment of the invention. The ingredients are as follows:

| Ingredient | % (w/v) |
|---|---|
| Sodium Hyaluronate, 1.0 million daltons | 0.02 to 0.3 |
| Sodium Chloride | 0.39 |
| Boric Acid | 0.6 |
| Sodium Borate Decahydrate | 0.035 |
| Potassium Chloride | 0.14 |
| Calcium Chloride, Dihydrate | 0.006 |
| Magnesium Chloride.6H₂O | 0.006 |
| Purite (stabilized oxy-chloro complex) | 0.005 |
| Sodium Hydroxide 1N NF | 7.2 (pH adjust) |
| Hydrochloric Acid 1N NF | 7.2 (pH adjust) |
| Purified Water | QS |

The balanced salts are dissolved in purified water followed by dissolution of the boric acid, sodium borate, and sodium hyaluronate. The pH is adjusted with base (1N sodium hydroxide) or acid (hydrochloric acid 1N) to 7.2 followed by the addition of purite. If necessary the pH is adjusted again and the solution adjusted to the final volume. The product is filled into teal bottles for light protection.

### EXAMPLE 2

### Stability Testing of Preferred Formulations

The stability of the following formulations were evaluated.

| | Formula A | Formula B |
|---|---|---|
| Ingredient | % (w/v) | % (w/v) |
| Sodium Hyaluronate, 1.0 million | 0.10 | 0.15 |
| daltons | | |
| Sodium Chloride Ph Eur USP | 0.39 | 0.39 |
| Boric Acid Ph Eur NF | 0.60 | 0.60 |
| Sodium Borate Decahydrate NF | 0.035 | 0.035 |
| Potassium Chloride USP | 0.14 | 0.14 |
| Calcium Chloride, Dihydrate USP | 0.006 | 0.006 |
| Magnesium Chloride Hexahydrate USP | 0.006 | 0.006 |
| Stabilized oxy-chloro complex | 0.005 | 0.005 |
| Sodium Hydroxide 1N NF | 7.2 (pH adjust) | 7.2 (pH adjust) |
| Hydrochloric Acid 1N NF | 7.2 (pH adjust) | 7.2 (pH adjust) |
| Purified Water | QS | QS |

The formulations were filled into 6-ml and 15-ml teal LDPE bottles. The 6-ml bottles contained 2-ml of each formulation while the 15-ml bottles contain 12-ml of each formulation. The bottles were stored at the following temperatures:

| Temperature (° C.) | Percent Relative Humidity |
|---|---|
| 25° C. ± 2° C. | 40% ± 5% |
| 30° C. ± 2° C. | 60% ± 5% |
| 37° C. ± 2° C. (for sterility testing only) | |
| 40° C. ± 2° C. | 20% ± 5% |

Two bottles of each configuration were tested for physical appearance, pH, potential chlorine dioxide, sodium hyaluronate concentration, osmolality, viscosity, visible light transmittance, sterility, and PET.

The formulations are stable for at least 24 months when stored at room temperature. This is based on the projections calculated from data obtained from product stored for nine months stored at 40° C. This is an improvement over the prior art, in that most sodium hyaluronate solutions on the market as viscoelastics for surgery require storage at refrigerated conditions due to stability problems.

### EXAMPLE 3

### Stability Testing of Formulations with and without Stabilized Oxy-Chloro Complex

The stability of the following formulations were evaluated.

| | Formula 1 | Formula 2 |
|---|---|---|
| Ingredient | % (w/v) | % (w/v) |
| Sodium Hyaluronate, 810,000 daltons | 0.10 | 0.10 |
| Sodium Chloride Ph Eur USP | 0.42 | 0.42 |
| Boric Acid Ph Eur NF | 0.60 | 0.60 |
| Sodium Borate Decahydrate NF | 0.035 | 0.035 |
| Potassium Chloride USP | 0.14 | 0.14 |
| Calcium Chloride, Dihydrate USP | 0.006 | 0.006 |
| Magnesium Chloride Hexahydrate USP | 0.006 | 0.006 |
| Stabilized Oxy-chloro Complex (Purite) | 0.005 (50 ppm) | — |
| Sodium Hydroxide 1N NF | 7.2 (pH adjust) | 7.2 (pH adjust) |
| Hydrochloric Acid 1N NF | 7.2 (pH adjust) | 7.2 (pH adjust) |
| Purified Water | QS | QS |

The formulations were identical except that Formula 2 did not contain stabilized oxy-chloro complex. Samples of each formula were stored at 25° C., 40° C., and 60° C., for 12, 3 and 2 months respectively. At each time point viscosity was measured. As discussed above, one of skill in the art would expect that the formula containing stabilized oxy-chloro complex would decrease in viscosity much faster than the formula without purite. As Table I illustrates, a direct comparison of the two formulas demonstrated that the viscosity of the formula containing stabilized oxy-chloro complex was surprisingly similar to the formula without purite. In fact, the initial decrease from the zero time point to the one month time point is much lower in Formula 1 than in Formula 2.

TABLE I
Viscosity (cps)

| | 25° C. | 25° C. | 40° C. | | | |
|---|---|---|---|---|---|---|
| | Formula | Formula | Formula | 40° C. | 60° C. | 60° C. |
| Month | 1 | 2 | 1 | Formula 2 | Formula 1 | Formula 2 |
| 0 | 6.60 | 8.50 | 6.60 | 8.50 | 6.60 | 8.50 |
| 1 | 4.15 | 4.51 | 3.69 | 3.98 | 2.33 | 2.81 |
| 2 | 3.95 | 3.89 | 3.56 | 3.60 | 1.04 | 1.42 |
| 3 | 4.00 | 3.90 | 3.45 | 3.42 | | |
| 4 | 3.93 | 3.92 | | | | |
| 6 | 3.85 | 3.75 | | | | |
| 9 | 3.67 | 3.75 | | | | |
| 12 | 3.49 | 3.19 | | | | |

### EXAMPLE 4

### Clinical Studies

Clinical studies were performed comparing preferred formulas A and B of Example 2 to commercially available Refresh. Groups of approximately 15 study subjects were followed for each formulation studied. Dosing consisted of one to two drops of the test formulation in one eye of each study subject with the remaining eye receiving one to two drops of control solution. The subjects were evaluated prior to treatment for baseline levels, immediately after treatment and at 5, 15, 30, and 60 minutes post-treatment. Results were assessed by the mean change from baseline at each time point.

The following safety evaluations were performed during the study. Slit lamp examinations, including the assessment of corneal edema, corneal neovascularization, corneal staining, injection/bulbar hyperemia, and palpebral conjunctiva status, were recorded at baseline and at all follow-up periods. Study lens-corrected visual acuity were recorded at baseline and at all follow-up periods using the ETDRS (Early Treatment of Diabetic Retinopathy Study) measurement system. Adverse events were monitored at all follow-up periods.

In addition to safety evaluations the following evaluations and measurements were made during the study. Subject qualifications, demography, lens wear history, pre-study lens care history, and medications were determined at the initial visit only. Lens wear comfort, symptoms of discomfort, overall subjective vision quality, and general comments were measured for baseline and at all follow-up periods. Lens fit quality and tear interferometry (tear film break-up time on the front surface of the contact lens) were measured for baseline and at all follow-up periods excluding the immediate post-dosing visit. Subject status was measured for baseline and at all follow-up periods excluding the immediate post-dosing visit unless required. Exit status was measured at all follow-up visits. Product acceptability was determined at the last exam.

As illustrated in the following tables, the clinical studies demonstrate that preferred formulations provide an increased length of comfort effect after using drops, greater comfort at the end of the day, improved tear break-up time, and longer lens wearing time during the day due to the enhanced comfort provided when compared to Refresh.

Study subject were asked to rate the length of the comfort effect after using the rewetter drops at day 7 and day 30 visits. Subjects using Formulas A and B reported longer more comfortable lens wear than patients using Refresh. For example, at day 30 13% of subjects using Formula A and 22.7% of subjects using Formula B reported that they did not need additional drops to maintain the comfort effect as compared to 4.8% for Refresh users.

TABLE II
Rating of Length of Comfort Effect After
Using Drops Rated At Each Scheduled Visit

| Visit | | Formula A | Formula B | Refresh |
|---|---|---|---|---|
| Day 7 | N | 24 | 22 | 23 |
| | Less than 15 | 1 (4.2%) | 1 (4.5%) | 2 (8.7%) |
| | Minutes | | | |
| | 15 to 30 Minutes | 1 (4.2%) | 2 (9.1%) | 2 (8.7%) |
| | >30 Minutes to 60 | 0 (0.0%) | 2 (9.1%) | 1 (4.3%) |
| | Minutes | | | |
| | >60 Minutes to 2 | 9 (37.5%) | 5 (22.7%) | 2 (8.7%) |
| | hours | | | |
| | >2 hours | 8 (33.5%) | 9 (40.9%) | 13 (56.5%) |
| | Not needed for | 5 (20.8%) | 3 (13.6%) | 3 (13.0%) |
| | Additional Drops | | | |
| Day 30 | N | 23 | 22 | 21 |
| | Less than 15 | 2 (8.7%) | 0 (0%) | 2 (9.5%) |
| | Minutes | | | |
| | 15 to 30 Minutes | 2 (8.7%) | 2 (9.1%) | 2 (9.5%) |
| | >30 Minutes to 60 | 0 (0%) | 2 (9.1%) | 1 (4.8%) |
| | Minutes | | | |
| | >60 Minutes to 2 | 7 (30.4%) | 4 (18.2%) | 4 (19.0%) |
| | hours | | | |
| | >2 hours | 9 (39.1%) | 9 (40.9%) | 11 (52.4%) |
| | Not needed for | 3 (13.0%) | 5 (22.7%) | 1 (4.8%) |
| | Additional Drops | | | |

Lens wear comfort at the end of each day were measured at day 0 for baseline, day 7 and day 30. Comfort scores were measured on a scale of 0 to 10 (from ‘lens cannot be tolerated’ to ‘lens cannot be felt’). Table III illustrates that formulas A and B provided a greater increase in comfort from baseline to day 30 when compared to Refresh.

TABLE III
Lens Wear Comfort at End-of-Day

| Visit | | Formula A | Formula B | Refresh |
|---|---|---|---|---|
| Baseline | N | 24 | 23 | 22 |
| | Mean | 7.3 | 7.5 | 7.2 |
| | SD | 1.55 | 1.54 | 1.71 |
| | Median | 8 | 8 | 7 |
| | Min | 5 | 5 | 5 |
| | Max | 10 | 10 | 10 |
| Day 7 | N | 24 | 22 | 22 |
| | Mean | 7.5 | 7.8 | 7.6 |
| | SD | 1.64 | 1.87 | 1.59 |
| | Median | 8 | 8 | 8 |
| | Min | 4 | 5 | 3 |
| | Max | 10 | 10 | 10 |
| Day 30 | N | 23 | 22 | 21 |
| | Mean | 7.6 | 8.2 | 7.4 |
| | SD | 1.38 | 1.32 | 1.89 |
| | Median | 8 | 8 | 7 |
| | Min | 5 | 5 | 4 |
| | Max | 10 | 10 | 10 |

Tear Break-Up time with lenses on was reported at each visit. The tear-break up time (TBUT) was measured at day 0 for baseline, and at days 7 and 30. Table IV illustrates that Formulas A and B showed improved or lengthened Tear Break-up time from baseline to day 30 as compared to Refresh. The change in tear break-up time for Formulas A and B from baseline to day 30 was an increase of 1.87 for Formula A and 3.06 for Formula B. Conversely, Refresh showed a decrease of 0.52 from baseline to day 30.

TABLE IV
Tear Break-up Time (in Seconds) with Lenses on

| Visit | | Formula A | Formula B | Refresh |
|---|---|---|---|---|
| Baseline | N | 24 | 23 | 23 |
| | Mean | 15.00 | 13.17 | 14.00 |
| | SD | 9.250 | 10.080 | 8.475 |
| | Median | 11 | 10 | 10 |
| | Min | 5 | 3 | 6 |
| | Max | 38 | 40 | 37 |
| Day 7 | N | 24 | 22 | 23 |
| | Mean | 16.00 | 14.36 | 13.52 |
| | SD | 9753 | 9.820 | 8.223 |
| | Median | 13 | 10 | 10 |
| | Min | 5 | 5 | 3 |
| | Max | 36 | 40 | 39 |
| Day 30 | N | 23 | 22 | 21 |
| | Mean | 16.87 | 16.23 | 13.48 |
| | SD | 10.248 | 10.506 | 8.256 |
| | Median | 12 | 13 | 10 |
| | Min | 7 | 3 | 4 |
| | Max | 45 | 40 | 38 |

Study subjects were asked to rate the change in lens wearing time since starting the study as compared to before the study. Ratings were taken at day 7 and 30. Table V illustrates that Formulas A and B increased wearing time by 21.7% and 18.2% respectively as compared to a 9.5% increase for Refresh.

TABLE V
Rating of Lens Wearing Time Since Starting
Study to Before Starting Study

| Visit | | Formula A | Formula B | Refresh |
|---|---|---|---|---|
| Day 7 | N | 24 | 22 | 23 |
| | Increased a Lot | 3 (12.5%) | 0 (0%) | 0 (0%) |
| | Increased Somewhat | 2 (8.3%) | 4 (18.2%) | 3 (13.0%) |
| | Not Changed | 18 (75.0%) | 18 (81.8%) | 19 (82.6%) |
| | Decreased | 0 (0%) | 0 (0%) | 0 (0%) |
| | Somewhat | | | |
| | Decreased a Lot | 1 (4.2%) | 0 (0%) | 1 (4.3%) |
| | Missing | 0 (0%) | 0 (0%) | 0 (0%) |
| Day 30 | N | 23 | 22 | 21 |
| | Increased a Lot | 1 (4.3%) | 0 (0%) | 0 (0%) |
| | Increased Somewhat | 4 (17.4%) | 4 (18.2%) | 2 (9.5%) |
| | Not Changed | 18 (78.3%) | 18 (81.8%) | 19 (90.5%) |
| | Decreased | 0 (0%) | 0 (0%) | 0 (0%) |
| | Somewhat | | | |
| | Decreased a Lot | 0 (0%) | 0 (0%) | 0 (0%) |
| | Missing | 0 (0%) | 0 (0%) | 0 (0%) |

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

1-102. (canceled)

103. A stable ophthalmic composition which is comfortable to the human eye comprising:
about 0.1% to about 0.6% w/v hyaluronic acid; and
about 0.0020% to about 0.02% w/v stabilized oxy-chloro complex;
wherein said composition increases tear film breakup time.

104. The stable ophthalmic composition of claim 103, wherein said hyaluronic acid has a molecular weight of about 800,000 to about 1,750,000 daltons.

105. The stable ophthalmic composition of claim 103, wherein the concentration of said hyaluronic acid is about 0.2% to about 0.4% w/v.

106. The stable ophthalmic composition of claim 103, wherein the concentration of said stabilized oxy-chloro complex is about 0.003% to about 0.01% w/v.

107. The stable ophthalmic composition of claim 103 further comprising a boric acid/borate buffer to maintain a pH of about 6.0 to about 9.0.

108. The stable ophthalmic composition of claim 107, wherein the pH of said composition is about 6.8 to about 8.0.

109. The stable ophthalmic composition of claim 108, wherein the pH of said composition is about 7.0 to about 7.4.

110. The stable ophthalmic composition of claim 103, further comprising balanced salts.

111. The stable ophthalmic composition of claim 110, wherein said balanced salts comprise NaCl, KCl, CaCl₂, and MgCl₂.

112. The stable ophthalmic composition of claim 110, wherein the balanced salts provide a composition osmolality of about 140 to about 400 mOsm/kg.

113. The stable ophthalmic composition of claim 112, wherein the balanced salts provide a composition osmolality of about 240 to about 330 mOsm/kg.

114. The stable ophthalmic composition of claim 103, further comprising about 0.05 to about 1% polyol demulcent.

115. The stable ophthalmic composition of claim 114, wherein the polyol demulcent is selected from the group consisting of glycerin, polyethylene glycol 300, polyethylene glycol 400, polysorbate 80 and propylene glycol.

116. The stable ophthalmic composition of claim 103 further comprising about 0.2 to about 2.5% cellulose derivative demulcent.

117. The stable ophthalmic composition of claim 116 wherein the cellulose derivative demulcent is selected from the group consisting of carboxymethylcellulose sodium, hydroxyethyl cellulose, hydroxypropyl methylcellulose, and methylcellulose.

118. A method of treating dry eye in a manner which is comfortable to the human eye comprising:
instilling a stable ophthalmic composition into the human eye;
wherein said stable ophthalmic composition comprises:
about 0.1% to about 0.6% w/v hyaluronic acid; and
about 0.0020% to about 0.02% w/v stabilized oxy-chloro complex.

119. The method of claim 118, wherein a contact lens is present in said human eye.

120. The method of claim 118, wherein said hyaluronic acid has a molecular weight of about 800,000 to about 1,750,000 daltons.

121. The method of claim 118, wherein the concentration of said hyaluronic acid is about 0.2% to about 0.4% w/v.

122. The method of claim 118, wherein the concentration of said stabilized oxy-chloro complex is about 0.003% to about 0.01% w/v.

123. The method of claim 118, wherein said composition further comprises a boric acid/borate buffer to maintain a pH of about 6.0 to about 9.0.

124. The method of claim 123, wherein the pH of said composition is about 6.8 to about 8.0.

125. The method of claim 124, wherein the pH of said composition is about 7.0 to about 7.4.

126. The method of claim 118, wherein said stable ophthalmic composition further comprises balanced salts.

127. The method of claim 126, wherein said balanced salts comprise NaCl, KCl, CaCl₂, and MgCl₂.

128. The method of claim 126, wherein the balanced salts provide a composition osmolality of about 140 to about 400 mOsm/kg.

129. The method of claim 128, wherein the balanced salts provide a composition osmolality of about 240 to about 330 mOsm/kg.

130. The method of claim 118, wherein said stable ophthalmic composition further comprises about 0.2 to about 1% polyol demulcent.

131. The method of claim 130, wherein the polyol demulcent is selected from the group consisting of glycerin, polyethylene glycol 300, polyethylene glycol 400, polysorbate 80 and propylene glycol.

132. The method of claim 118 wherein said stable ophthalmic composition further comprises about 0.2 to about 2.5% cellulose derivative demulcent.

133. The method of claim 132 wherein the cellulose derivative demulcent is selected from the group consisting of carboxymethylcellulose sodium, hydroxyethyl cellulose, hydroxypropyl methylcellulose, and methylcellulose.
